(19) United States
(12) Patent Application Publication
(10) Pub. No.: US 2022/0405933 A1
(43) Pub. Date: Dec. 22, 2022

(54) SYSTEMS, METHODS, AND APPARATUSES FOR IMPLEMENTING ANNOTATION-EFFICIENT DEEP LEARNING MODELS UTILIZING SPARSELY-ANNOTATED OR ANNOTATION-FREE TRAINING

(71) Applicant: Arizona Board of Regents on behalf of Arizona State University, Scottsdale, AZ (US)

(72) Inventors: Nima Tajbakhsh, Los Angeles, CA (US); Jianming Liang, Scottsdale, AZ (US)

(21) Appl. No.: 17/843,817

(22) Filed: Jun. 17, 2022

Related U.S. Application Data
(60) Provisional application No. 63/212,431, filed on Jun. 18, 2021.

(51) Int. Cl. G06T 7/00; G06V 10/82; G06V 10/774; G16H 50/20

(52) U.S. Cl. CPC G06T 7/0014; G06V 10/82; G06V 10/7747; G16H 50/20

(57) ABSTRACT

Described herein are means for implementing annotation-efficient deep learning models utilizing sparsely-annotated or annotation-free training, in which trained models are then utilized for the processing of medical imaging. An exemplary system includes at least a processor and a memory to execute instructions for learning anatomical embeddings by forcing embeddings learned from multiple modalities; initiating a training sequence of an AI model by learning dense anatomical embeddings from unlabeled date, then deriving application-specific models to diagnose diseases with a small number of examples; executing collaborative learning to generate pretrained multimodal models; training the AI model using zero-shot or few-shot learning; embedding physiological and anatomical knowledge; embedding known physical principles refining the AI model; and outputting a trained AI model for use in diagnosing diseases and abnormal conditions in medical imaging. Other related embodiments are disclosed.

### CLAIM OF PRIORITY

This application is related to, and claims priority to, the provisional U.S. Patent Application entitled “SYSTEMS, METHODS, AND APPARATUSES FOR IMPLEMENTING ANNOTATION-EFFICIENT DEEP LEARNING MODELS UTILIZING SPARSE OR EMPTY TRAINING DATASETS,” filed on Jun. 18, 2021, having an application number of 63/212,431 and Attorney Docket No. 37684.664P, the entire contents of which are incorporated herein by reference as though set forth in full.

### GOVERNMENT RIGHTS AND GOVERNMENT AGENCY SUPPORT NOTICE

This invention was made with government support under R01 HL128785 awarded by the National Institutes of Health. The government has certain rights in the invention.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD

Embodiments of the invention relate generally to the field of medical imaging and analysis using convolutional neural networks for the classification and annotation of medical images, and more particularly, to systems, methods, and apparatuses for implementing annotation-efficient deep learning models utilizing sparsely-annotated or annotation-free training, in which trained models are then utilized for the processing of medical imaging.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to embodiments of the claimed inventions.

Machine learning models have various applications to automatically process inputs and produce outputs considering situational factors and learned information to improve output quality. One area where machine learning models, and neural networks in particular, provide high utility is in the field of processing medical images.

Within the context of machine learning and with regard to deep learning specifically, a Convolutional Neural Network (CNN, or ConvNet) is a class of deep neural networks, very often applied to analyzing visual imagery. Convolutional Neural Networks are regularized versions of multilayer perceptrons. Multilayer perceptrons are fully connected networks, such that each neuron in one layer is connected to all neurons in the next layer, a characteristic which often leads to a problem of overfitting of the data and the need for model regularization. Convolutional Neural Networks also seek to apply model regularization, but with a distinct approach. Specifically, CNNs take advantage of the hierarchical pattern in data and assemble more complex patterns using smaller and simpler patterns. Consequently, on the scale of connectedness and complexity, CNNs are on the lower extreme.

Heretofore, self-supervised learning has been sparsely applied in the field of medical imaging. Nevertheless, there is a massive need to provide automated analysis to medical imaging with a high degree of accuracy so as to improve diagnosis capabilities, control medical costs, and to reduce workload burdens placed upon medical professionals.

Not only is annotating medical images tedious and time-consuming, but it also demands costly, specialty-oriented expertise, which is not easily accessible.

The present state of the art may therefore benefit from the systems, methods, and apparatuses for implementing annotation-efficient deep learning models utilizing sparsely-annotated or annotation-free training, in which trained models are then utilized for the processing of medical imaging, as is described herein.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1 illustrates the major areas of focus in the context of annotation-efficient deep learning, according to described embodiments;

FIG. 2 shows a diagrammatic representation of a system within which embodiments may operate, be installed, integrated, or configured, in accordance with one embodiment;

FIG. 3 illustrates a diagrammatic representation of a machine in the exemplary form of a computer system, in accordance with one embodiment; and

FIG. 4 depicts a flow diagram illustrating a method for implementing annotation-efficient deep learning models utilizing sparsely-annotated or annotation-free training, in which trained models are then utilized for the processing of medical imaging, in accordance with disclosed embodiments.

### DETAILED DESCRIPTION

Described herein are systems, methods, and apparatuses for implementing annotation-efficient deep learning models utilizing sparsely-annotated or annotation-free training, in which trained models are then utilized for the processing of medical imaging.

Annotation-efficient deep learning refers to methods and practices that yield high-performance deep learning models without the use of massive carefully labeled training datasets. This paradigm has recently attracted attention from the medical imaging research community because (1) it is difficult to collect large, representative medical imaging datasets given the diversity of imaging protocols, imaging devices, and patient populations, (2) it is expensive to acquire accurate annotations from medical experts even for moderately-sized medical imaging datasets, and (3) it is infeasible to adapt data-hungry deep learning models to detect and diagnose rare diseases whose low prevalence hinders data collection.

The challenge of annotation-efficient deep learning has been approached from various angles in the medical imaging literature, however, the relevant publications are scattered across numerous sources and there exist many gaps that require further research.

The described embodiments address these deficiencies by introducing innovative improvements to the known state-of-the-art methodologies spanning major topics of annotation-efficient deep learning.

Many state of the art techniques fall into the categories of leveraging unannotated data and utilizing annotations efficiently. Popular approaches include zero/few-shot learning, domain adaptation, learning from weak and noisy labels, and synthetic data augmentation. Each of these present potential opportunities for innovation as described below as well as opportunity for future research and thus further enhancement.

In the following description, numerous specific details are set forth such as examples of specific systems, languages, components, etc., in order to provide a thorough understanding of the various embodiments. It will be apparent, however, to one skilled in the art that these specific details need not be employed to practice the embodiments disclosed herein. In other instances, well known materials or methods have not been described in detail in order to avoid unnecessarily obscuring the disclosed embodiments.

In addition to various hardware components depicted in the figures and described herein, embodiments further include various operations which are described below. The operations described in accordance with such embodiments may be performed by hardware components or may be embodied in machine-executable instructions, which may be used to cause a specialized and special-purpose processor having been programmed with the instructions to perform the operations described herein. Alternatively, the operations may be performed by a combination of hardware and software. In such a way, the embodiments of the invention provide a technical solution to a technical problem.

Embodiments also relate to an apparatus for performing the operations disclosed herein. This apparatus may be specially constructed for the required purposes, or it may be a special purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

The algorithms and displays presented herein are not inherently related to any particular computer or other apparatus. Various customizable and special purpose systems may be used with programs in accordance with the teachings herein, or it may prove convenient to construct more specialized apparatus to perform the required method steps. The required structure for a variety of these systems will appear as set forth in the description below. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems which are not directly discussed.

In addition to various hardware components depicted in the figures and described herein, embodiments further include various operations which are described below. The operations described in accordance with such embodiments may be performed by hardware components or may be embodied in machine-executable instructions, which may be used to cause a special-purpose processor programmed with the instructions to perform the operations. Alternatively, the operations may be performed by a combination of hardware and software, including software instructions that perform the operations described herein via memory and one or more processors of a computing platform.

Acquiring Annotations Efficiently:

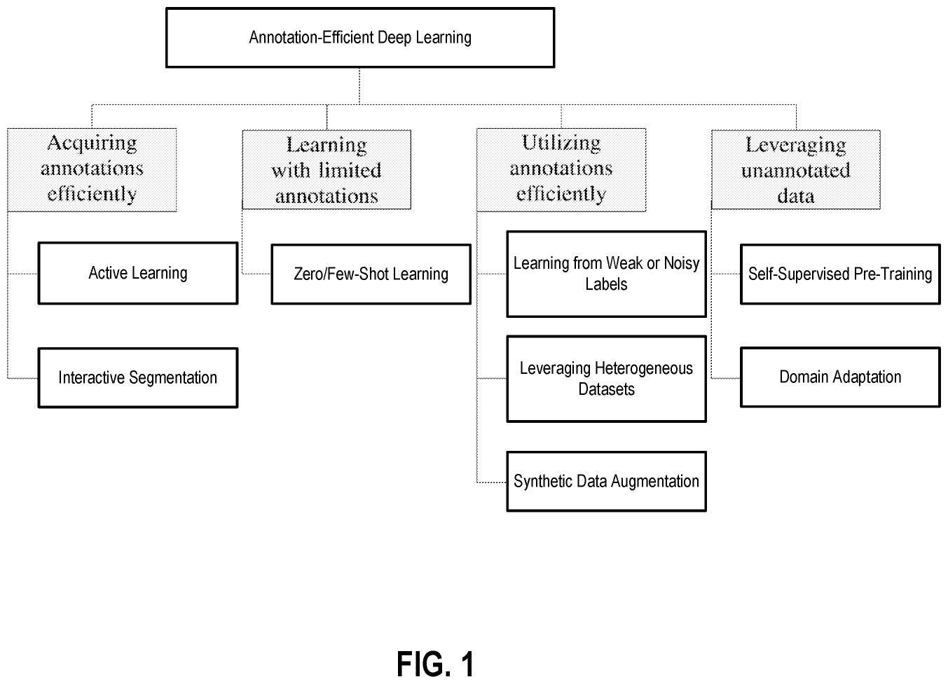

FIG. 1 illustrates the major areas of focus in the context of annotation-efficient deep learning, according to described embodiments.

Active learning and interactive segmentation are two mechanisms which may be used for the purposes of acquiring annotations in a budget-efficient manner. The former determines which data samples should be annotated, whereas the latter shortens the annotation session.

The two approaches are complementary and they enable substantial reductions in annotation time and cost.

Active Learning:

Active learning aims to select the most informative and representative samples for experts to annotate, thereby minimizing the total number of samples that must be annotated to train performant models. The effectiveness of an active learning method largely depends on its criteria for determining the informativeness and representativeness of an unlabeled sample. The more uncertain the sample's prediction, the more information its label offers.

Minimizing the number of annotated samples requires that the labeled samples be distinct from one another. Therefore, uncertainty and diversity are two natural metrics for informativeness and representativeness.

For instance, one approach operates on the principle that duplicating uncertain samples in the labeled training dataset helps decrease the overall model uncertainty, and they regulate the amount of duplication based on mutual information between data pairs of the training and unlabeled pool.

Alternatively, another approach utilizes a self-supervised method for training a classifier to select informative samples based on saliency maps that embody both uncertainty and diversity.

Interactive Segmentation:

Smart interactive segmentation tools play an essential role in reducing the manual burden of producing high-quality annotated data. Innovative techniques include the exploration of boundary-aware multi-agent reinforcement learning to interactively and iteratively refine 3D image segmentations. The approach is able to combine different types of user interactions, such as point clicks and scribbles, via an efficient “super-voxel-clicking” design.

Further innovation includes the use of few-shot learning to arrive at better medical image segmentation using only limited supervision. The approach has the potential to reduce the annotation burden and fix some common issues in few-shot learning methods by enabling the user to make minor corrections interactively. For instance, the methodology is specially configurable via executing instructions to permit the initiation of a training sequence of an AI model by first learning of dense anatomical embeddings to train the AI model to mimic human abilities of learning to identify and diagnose certain diseases through the study of a small number of textbook examples. This is sometimes referred to as “few-shot” learning.

Few-Shot Learning:

In the context of AI disciplines, few-shot learning is the problem of making predictions based on a limited number of samples. Few-shot learning is different from standard supervised learning. With few-shot learning, the model does not affirmatively recognize the images in the training set and must therefore generalize to the test set given the absence of a large incoming curated training data set.

More specifically, few-shot learning refers to the problem of specially configuring an AI model to learn an underlying pattern in a data set from only a limited few training samples. Prior approaches require a large number of data samples, and as such, many deep learning solutions suffer from data hunger and extensively high computation time and resources. Furthermore, data is often not available due to not only the nature of the problem or privacy concerns but also the cost of data preparation. This is especially problematic in the context of analyzing medical imaging data. Data collection, preprocessing, and labeling are strenuous human tasks. Therefore, few-shot learning can drastically reduce the turnaround time of building machine learning applications.

Human cognitive sciences and developmental psychology teaches that humans have innate abilities, such as programmed instincts that appear at birth or in early childhood, that enable humans, even children, to think abstractly and flexibly. Studies show that humans can even perform classification tasks (e.g., recognize the subject of an image) after being exposed to very few training examples. For instance, a child that sees two cats and two dogs (e.g., is exposed to training data) will readily recognize a different and never before seen cat or dog with high accuracy.

Therefore, methodologies are described herein which operate to specially configure a system to train an AI model to “learn” suitable classification operations even when exposed to very few training examples, referred to as “few-shot” training. Approaches described herein therefore mimic the desirable human capability, but do so using very different approaches, such as through the generation and integration of supplemental training data that does not exist within an incoming data set presented to the AI model.

Learning with Limited Annotations:

As suggested above, the ability to analyze medical images with little or no training data is highly desirable. For example, one such innovative technique utilizes an unsupervised deep learning method for multi-contrast MR image registration, using a coarse-to-fine network architecture consisting of affine coarse and deformable fine transformations to achieve end-to-end registration.

For the task of semantic segmentation, compiling large-scale (non-synthetic) medical image datasets with pixel-level annotations is time-consuming and often prohibitively expensive. Moreover, it may be impossible to balance all the relevant classes in the training set. Although semi-supervised approaches aim to relax the level of supervision to bounding boxes and image-level tags, these models still require copious training samples and are prone to sub-optimal performance on unseen classes. However, very often the training data sets needed to adequately train such models is simply sparse or entirely non-existent.

Zero/Few-Shot Learning: By contrast, the few-shot learning paradigm as proposed herein attempts to utilize a small quantity of annotated “support” samples to learn representations of unseen classes, denoted “query” samples.

The few-shot learning paradigm was initially focused on classification but then later applied to segmentation. In the context of volumetric ultrasound segmentation, a decremental update of the objective function may be utilized to guide model convergence in the absence of copious annotated data. With such a technique, the update strategy therefore switches from weakly supervised training to a few-shot setting. Also in the context of volumetric segmentation, such as with reference to medical imaging of the human heart, a few-shot learning framework is proposed that combines ideas from semi-supervised learning and self-training.

The key to success for such a technique lies in cascaded learning; specifically, in the selection and evolution of high-quality pseudo labels produced by an auto-encoder network that learns shape priors.

Interactive learning may further be introduced into the few shot learning strategy for image segmentation. With such an approach, an expert indicates error regions as an error mask that corrects the predicted segmentation mask from which the network learns to produce better segmentations.

Thus, a potential zero-shot learning strategy for the diagnosis of chest radiographs includes leveraging multi-view semantic embedding and incorporating self-training to deal with noisy labels.

A unified framework for generalized low-shot medical image segmentation may further be utilized to deal with both the issue of data scarcity as well as the problem of lack of annotations, which is usually the situation with respect to rare diseases.

Using distance metric learning, a model is trained to learn a multimodal mixture representation for each category, which then helps to effectively avoid over-fitting the extremely limited data.

Finally, to facilitate image segmentation a traditional model-based contour segmentation method is combined with deep learning methods to accrue the benefits of both approaches. Such an approach thus formulates anatomy segmentation as a contour evolution process, using Active Contour Models whose evolution is governed by graph convolutional networks. Such a model requires only one labeled image exemplar and supports human-in-the loop editing. This approach for one-shot learning thus leverages additional unlabeled data through loss functions that measure the global shape and appearance consistency of the contours.

Utilizing Annotations Efficiently:

Acquiring additional strongly-annotated data is arguably the best approach to improving deep learning models, but this practice may not always be feasible due to limited budgets or shortages of medical expertise. Consequently, one may resort to using annotations that are cheaper or faster to obtain. The resulting weak and noisy annotations require proper learning methodologies. Another approach to this challenge is to leverage related annotated datasets, which can increase the effective size of training sets. Synthetic data augmentation is yet another possible approach by which the training sets are amplified by creating artificial examples and corresponding annotations.

Learning From Weak or Noisy Labels: Enabling learning from weak or noisy labels can be especially useful for medical imaging as the collection of quality labels is time-consuming, cumbersome, and often requires expert knowledge.

It is notable that recent innovations squarely seek to leverage the availability of weak or noisy labels that are routinely recorded during clinical workflows, such as measurements taken by clinicians (e.g., such as the case with “RECIST” or “Response Evaluation Criteria In Solid Tumors”) as well as the case with image-level labels provided in clinical reporting.

These opportunities are addressed by exploiting both “weak” labels (e.g., image-level labels instead of pixel/voxel-wise labels) or “noisy” labels transferred from other modalities, and demonstrated promise by effectively combining supervision from such weak or noisy signals to train medical imaging models. Thus, one viable research direction is the utilization of weak image-level labels. For example, a self-supervised attention mechanism may be utilized to learn from image-level labels for pediatric bone age assessment.

One approach thus seeks to exploit weak supervision from image-level labels to train abnormality localization models for chest X-ray diagnosis or to utilize image-level labels from mammography to detect abnormalities in a weakly self-supervised fashion. Still another solution may be to combine weakly-supervised and semi-supervised learning to train models from both image-level and densely-labeled images. Both attention guidance and multiple-instance learning are utilized to effectively learn from both types of annotated data for the purposes of adenocarcinoma prediction in abdominal CT. Yet another research direction is to explore the use of point annotations for weakly supervised cell segmentation in microscopy images. Using one point per cell, segmentation models are trained and perform comparably to models trained on dense annotation maps. The utilization of noisy labels is further possible in which the use weak-supervision for automated vessel detection in ultra wide-field fundus images. Such an approach exploits a deep neural network pre-trained on a different imaging modality, fluorescein angiography, together with multi-model registration to iteratively train their model and simultaneously refine the registration.

Leveraging Heterogeneous Datasets: Another promising approach to mitigating annotation costs in medical imaging is to leverage multiple heterogeneous datasets. These datasets might have been acquired for other purposes, but they can be combined to build more robust models.

For instance, one technique is to train a lesion detection ensemble from multiple heterogeneous lesion datasets in a multi-task approach. The ensemble is utilized for proposal fusion and missing annotations are mined from partially labeled datasets to improve the overall detection sensitivity.

Another technique is to exploit intra-domain and inter-domain knowledge to improve cardiac segmentation models. When such a method is evaluated on a multi-modality dataset, there are demonstrable improvements over previous semi-supervised and domain adaptation methods. Both techniques are promising with regard to the future development of AI models as it is natural to combine information about human anatomy acquired for different purposes. These approaches may enable the leveraging of features between datasets in order to build more powerful and robust models while drastically reducing the cost of curating task-specific datasets.

Synthetic Data Augmentation:

Synthetic data augmentation aims to generate artificial yet realistic-looking images, thereby enabling the construction of large and diverse datasets. This practice is particularly desirable for organ segmentation where acquiring large labeled datasets is expensive and time-consuming and, even more importantly, for lesion segmentation where data collection is hindered by the low prevalences of underlying diseases and conditions.

Techniques demonstrating the addition of synthetic data to real data are shown to either improve segmentation performance or enable a comparable level of performance while reducing the need for real annotated data.

Such techniques cover various medical imaging modalities, including MR scans, CT scans, ultrasound images, microscopic images, and retinal images. The methods differ in the training data required to synthesize images and their corresponding segmentation masks. Relying on variants of the CycleGAN, certain techniques exploit image synthesis for the task of image segmentation, but take different measures to circumvent the use of segmentation masks from the target domain.

Specifically, one technique requires the use of a pre-existing 3D shape model of the heart, another requires segmentation masks from a domain only related to the target domain, and yet another technique relies on a handcrafted model to generate synthetic COVID lesions.

Two other techniques do require segmentation masks from the desired domain in order to generate synthetic data. In particular, one technique generates synthetic segmentation masks through an active appearance model, which is trained using segmentation masks from the target domain, whereas the other technique relies upon brain atlases, which makes the method suitable for applications where high-quality atlases are available for training.

Despite the promising performances of these techniques, the requirement for atlases or shape and appearance models of the target diseases or organs may nonetheless limit applicability where training data is sparse or non-existent.

Leveraging Unannotated Data:

Unannotated images contain a wealth of knowledge that can be leveraged in various settings, such as self-supervised learning and unsupervised domain adaptation. The former utilizes unannotated data to pre-train the model weights, whereas the latter leverages unannotated data from a target domain to mitigate the distribution shifts between the training and test datasets.

Self-Supervised Learning:

Self-supervised learning has recently gained prominence for its capacity to learn generalizable and transferable representations without the use of any expert annotation. The idea is to pretrain models on pretext tasks (e.g., rotation, inpainting, and contrasting), where supervisory signals are automatically derived directly from the unlabeled data themselves (avoiding expert annotation costs), and then fine-tune (transfer) the pre-trained models in a supervised manner so as to achieve annotation efficiency for the target tasks (e.g., segmenting organs and localizing diseases).

Self-supervised learning leverages the underlying structures and intrinsic properties of the unlabeled data, a feature that is particularly attractive in medical imaging. To take advantage of the special property of pathology, one approach is to design pathology-specific pretext tasks and demonstrate annotation efficiency for the target domain in the small data regime. To exploit the semantics of anatomical patterns embedded in medical images, such a technique may pre-train a model using chest CT which has been experimentally demonstrated across-domain capability to contrast-enhanced CT and MRI. Use of such a technique may further operate as an add-on strategy, as the method complements existing self-supervised methods, including inpainting, context restoration, rotation, and models genesis, thus boosting performance of such techniques.

Domain Adaptation:

Deep learning models struggle to generalize when the target domain exhibits a data distribution shift with respect to the source dataset used for training. This challenge is even more pronounced in the field of medical imaging where variations in ethnicities, scanner devices, and imaging protocols lead to large data distribution shifts. Unsupervised domain adaptation has emerged as an effective approach to improving the tolerance of deep learning models to the distribution shifts in medical imaging datasets.

Six techniques address the distribution shift through an episodic training strategy where the training data of each episode are generated so as to mimic a distribution shift with respect to the original training dataset.

Five of the six techniques are shown to improve the CycleGAN in domain adaptation, whereas a sixth technique includes auxiliary segmentation and reconstruction tasks. One method exercises consistency regularization during training. A second introduces a self-attentive spatial normalization block in the generator networks. A third uses task-specific probabilities to focus the attention of the transformation module on more relevant regions. With each technique, domain adaptation is leveraged for various applications, including reorienting MR images, staining unstained white blood cell images, cross-modality heart chamber segmentation between unpaired MR and CT scans, nucleus detection in cross-modality microscopy images, lung nodule detection in cross-protocol CT datasets, and various medical vision applications in cross-domain retinal imaging.

New Innovations and Further Research Opportunities:

Notwithstanding the above described techniques, there still remains unanswered questions and unaddressed issues for which new innovations as described herein may be applied as well as questions and unaddressed issues that offer many enticing research opportunities.

Quantifying annotation efficiency: Annotations generally refer to the ground truth information that is used in training, validating, and testing models. In medical imaging, this information is mostly provided (subjectively) by experts, but it may also derive from objective conditions obtained through tests (e.g., the malignancy of a tumor) and from medical concepts (diseases and conditions) automatically extracted from clinical notes and diagnostic reports. In a broader sense in self-supervised learning, it could also be a part of the data that are to be predicted based on other parts of the data or the original data that are to be restored from their transformed versions.

According to certain embodiments, annotations are acquired at the patient, image, and pixel levels. Therefore, such annotations require different levels of effort/cost and offer different levels of semantics/power as supervisory signals in training models.

With regard to such annotations, a first method (A) is said to be more efficient than a second Method (B) if, compared with Method B, Method A (1) achieves better performance with equal annotation cost, (2) offers equal performance with lower annotation cost, or (3) provides equal performance with equal annotation cost, but reduces the training time.

Currently, the prevailing literature generally assumes the same annotation cost for each and every sample (e.g., patient, image, or lesion). The reality is quite different, however, as costs could vary dramatically from one sample to another. It is also important to understand the trade-off between annotation cost and supervisory power in different settings. For example, for proton therapy, a few lesions with carefully-delineated masks may have more supervisory power than many lesions coarsely bounded by boxes. There is a need for new concepts, algorithms, and tools to analyze annotation efficiency across different contexts.

Annotating patients efficiently: This matter is essential to medical image analysis, and resolving it can greatly accelerate the development of deep learning models. Four techniques are described, yet the issue is generally under-investigated. In particular, two techniques utilize active learning primarily focusing on the tasks of image segmentation and classification, leaving other important tasks such as lesion detection largely unexplored. This application bias is not peculiar; in fact, it applies to the entire medical imaging literature.

Possibly the issue is due to the fact that finding an optimal set of samples that are informative and representative is inherently difficult. With regard to interactive segmentation, two techniques show promising task-specific solutions. However, generic interactive segmentation tools remain difficult to build for medical imaging applications. The type of user interactions employed (points, scribbles, bounding boxes, polygons, etc.) are often based on the targeted anatomy, which tends to hinder generalization to new anatomical structures. Building more comprehensive datasets with which to train general-purpose interactive models on several target anatomies and/or imaging modalities may be a good way forward.

It would be desirable to develop tools that integrate active learning with interactive segmentation, as active learning can select the most important samples for annotation whereas interactive segmentation can shorten the annotation session for each sample. Active learning may be also embedded within interactive segmentation to suggest which parts of the image should be segmented first, thereby further accelerating the annotation process. Not only will such tools prove to be valuable for clinical purposes, but they will also be indispensable for building a massive, strongly-annotated dataset as essential infrastructure for research without which the annotation efficiency of a method can hardly be quantitatively determined or benchmarked.

Learning by zero/few shots: Certain disclosed techniques focus on zero-shot or few-shot learning for medical image classification and segmentation, reflecting the immediate need to progress beyond data-intensive machine learning methods. The promise of this technology is especially appealing in the healthcare domain where the collection of large and varied annotated datasets is difficult and sometimes hindered by regulations, a problem that is aggravated when studying rare diseases for which the acquisition of training examples becomes even more difficult.

Therefore, techniques are disclosed in which it is beneficial to consider the use of multi-modality information in designing few shot learning methods. For instance, according to certain embodiments, one may force the embeddings learned from multiple modalities (e.g., X-rays, CT, MRI, and reports) to be matched in a shared space, thereby encouraging collaborative learning via joint-supervision and cross-supervision.

Furthermore, analogous to the saying that training to identify counterfeit currency begins with studying genuine money, an effective approach to zero-shot or few-shot learning for diagnosing diseases and abnormal conditions in medical imaging may, according to such embodiments, start with learning dense (normal) anatomical embeddings. Successful zero-shot and few-shot learning will bring trained AI systems and models closer to human abilities, similar to the manner in which a clinician learns to identify/diagnose certain diseases by studying just a few textbook examples.

Synthesizing annotations: Artificially generating realistic-looking images with associated ground truth information helps relieve laborious human annotation and facilitates the creation of large datasets.

This is particularly attractive for image segmentation where acquiring carefully-delineated masks is tedious and time-consuming as well as for localization where collecting rare diseases and conditions is challenging. Hand-crafted or trained generative models have proven to be powerful in creating “realistic” images and videos.

Nevertheless, for the purposes of medical imaging, special attention and care must be given to potential artifacts.

Therefore, embedding physiological and anatomical knowledge as well as the physical principles of image modalities into the synthesis process may prove to be critical.

Innovating architectures: Recent advances in network architecture design have proven successful in natural language processing and computer vision. However, it is not clear whether these new architectures are more annotation-efficient than their predecessors. For instance, certain techniques show that transformer architectures as well as a new family of efficient models, called EfficientNet v2.0, benefit from a significantly larger version of ImageNet, which raises concerns about the annotation-efficiency of new architectures.

Further research is required to study the annotation-efficiency of such models, particularly in the context of medical imaging. It would be intriguing to investigate if, by reducing the need for annotated data, annotation-efficient methods can engender new architectural advancements. For instance, one proposed technique demonstrates that self-supervised training is effective in reducing the amount of annotated visual data required to train transformers. It is worth studying how such recent architectural advancements benefit from other annotation-efficient paradigms in the context of medical imaging.

Although the medical imaging community tends to adopt deep architectures developed for computer vision, given the differences between medical and natural images, and so as to maximize annotation utilization, it would be worth designing or automatically searching for architectures that exploit the particular opportunities of medical imaging for annotation efficiency.

Exploiting clinical information: Medical concepts (diseases and conditions) extractable from clinical notes and reports may be capitalized as (weak) patient-level annotation. Indeed, natural language processing (NLP) may be utilized to harvest image-level annotations from diagnostic reports in order to build medical imaging datasets and used to develop machine learning models, but the vast resources of clinical notes and diagnostic reports that are available in electronic health records of hospital systems have yet to be fully mined and well utilized.

Learning representations jointly from both images and texts (clinical notes and diagnostic reports) is therefore set forth as a novel innovation as well as a very active area of further research, although patient privacy and other regulatory constraints must be carefully considered.

Integrating data and annotation from multiple sources: Datasets created at different institutions tend to be annotated differently even when addressing the same clinical issue.

There remains a need for learning methods that can seamlessly integrate data and annotation from different sources. Federated learning (FL) has recently emerged as an effective solution for addressing the growing concerns about data privacy when integrating data and annotation from different providers. Federated learning trains a model using data from various sites without breaching patient privacy and other regulations, thereby making more data available for AI model development.

Federated learning has yet to seamlessly handle the unavoidable heterogeneity of data across different sites. Semi-supervised and unsupervised/self-supervised approaches have already been successfully used in the context of federated learning. Nevertheless, it will be interesting to see how other annotation-efficient methods can be combined with federated learning as well as how federated learning and other methods that integrate data and annotation from multiple sources can relieve the annotation burden.

Mining common knowledge: Currently, the dominant approach in deep learning—supervised learning—offers expert-level and sometimes even super-expert-level performance.

Models trained via supervised learning have also demonstrated remarkable capacity in knowledge transfer across domains, but at their core, they are trained to be “specialists” on (target) tasks that can be annotated by experts. There are many diseases and conditions in medical imaging as well as common medical knowledge that can hardly be annotated, even by willing experts.

Self-supervised learning has proven to be promising in training models to be “generalists” on various pretext tasks so as to reduce expert annotation effort on target tasks.

Typically, the semantics of expert-provided annotation is strong but narrow (task-specific), while that of machine-generated annotation in self-supervised learning is weak but general.

Fundamental to annotation efficiency is learning common generalizable knowledge. Therefore, self-supervised learning will inevitably overtake supervised learning in extracting generalizable and transferable knowledge; i.e., self-supervised representation learning followed by (supervised) transfer learning is poised to become the most practical paradigm towards annotation efficiency. This is particularly true for medical imaging, because medical images harbor rich semantics about human anatomy, thus offering a unique opportunity for deep semantic representation learning. And yet, harnessing the powerful semantics associated with medical images remains largely unexplored.

Furthermore, medical images are often augmented by clinical notes and reports, making it even more attractive to learn generic semantic representations jointly from both images and reports via self-supervision.

Minding common knowledge will prove to be impactful on learning by zero/few shots, accelerating model training (supervised, self-supervised, semi-supervised, unsupervised, federated, etc.), creating dense (normal) anatomical embeddings, characterizing diseases, their sub-types, and their semblances (false positives), and enhancing system performance and robustness.

Reusing knowledge in trained models: Research and development in deep learning across academia and industry have resulted in numerous models trained on various datasets in supervised, self-supervised, unsupervised, or federated manners for diverse clinical objectives. These trained models retain a large body of knowledge, and properly reusing this knowledge could reduce annotation efforts and accelerate training cycles, thereby increasing annotation efficiency.

However, current practice in reusing knowledge from (existing) pre-trained models for new tasks is very limited. Therefore, advanced methods are needed for transferring, reusing, and distilling the knowledge from pre-trained models as well as integrating the knowledge from multiple pre-trained models with the same or distinct architectures.

Demonstrating annotation efficiency in practice: Methods and techniques are being developed from various perspectives to circumvent the annotation dearth in medical imaging; their value and effectiveness needs to be quantitatively evaluated and benchmarked. This calls for an infrastructure of massive, strongly-annotated datasets in well-defined domains (e.g., such as pulmonary embolism, colon cancer, and cardiovascular disease), without which the annotation efficiency of a method cannot be adequately understood.

Given the above techniques and opportunities for further research, deep learning has dramatically transformed medical imaging, but annotation-efficient deep learning remains the yet to be realized Holy Grail of medical imaging.

It would therefore be highly beneficial for large-scale competitions and challenges to be organized which would in turn, encourage others to further refine and build upon the described methodologies as set forth herein as well as encourage the additional development of annotation-efficient methods and practices in the design and development of commercial products.

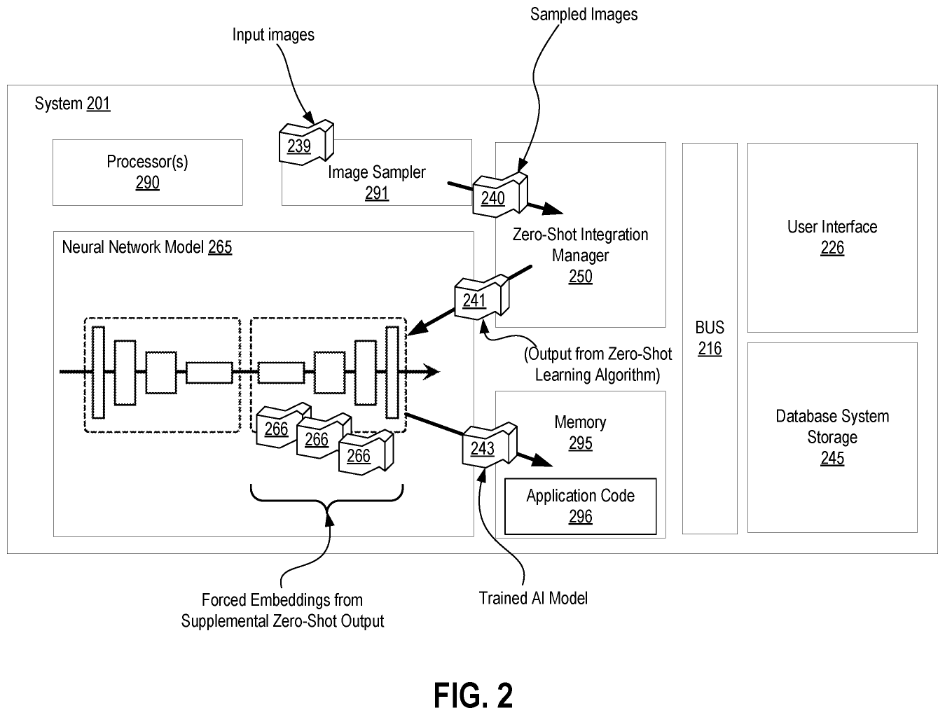

FIG. 2 shows a diagrammatic representation of a system 201 within which embodiments may operate, be installed, integrated, or configured. In accordance with one embodiment, there is a system 201 having at least a processor 290 and a memory 295 therein to execute implementing application code 296. Such a system 201 may communicatively interface with and cooperatively execute with the benefit of remote systems, such as a user device sending instructions and data, a user device to receive as an output from the system 201.

According to the depicted embodiment, the system 201, includes the processor 290 and the memory 295 to execute instructions at the system 201. The system 201 as depicted here is specifically customized and configured specifically to train a deep model to learn and integrate a defense mechanism into deep-learning-based AI model and system to implement annotation-efficient deep learning models utilizing sparse or empty training datasets, in which trained models are then utilized for the processing of medical imaging, in accordance with disclosed embodiments.

According to a particular embodiment, system 201 is specifically configured to execute instructions via the processor for force embedding supplemental zero-shot learning output 266 from the zero-shot integration manager 250 when training an AI model to generate and output a trained AI model 243 by executing the following operations: executing instructions via the processor 290 for embedding physiological and anatomical knowledge into the training of an AI model 243; embedding known physical principles of image modalities from input images 239 via the image sampler 291 into a synthesis process for refining the AI model 243; in which the trained AI model 243 is trained for diagnosing diseases and abnormal conditions in medical imaging. Additional operations include executing instructions via the processor 290 for learning representations jointly from both images (e.g., input images 239) and texts (e.g., output from the zero-shot learning algorithm 241 as provided by the zero-shot integration manager 250) including one or more of clinical notes and diagnostic reports; in which the trained AI model 243 is trained for diagnosing diseases and abnormal conditions in medical imaging. As depicted, the neural network 265 operates to integrate provided information including input image data 239 and output from the zero-shot learning algorithm and the forced embeddings from the supplemental zero-shot output 266 to generate the trained AI model 243.

According to another embodiment of the system 201, a user interface 226 communicably interfaces with a user client device remote from the system and communicatively interfaces with the system via a public Internet.

Bus 216 interfaces the various components of the system 201 amongst each other, with any other peripheral(s) of the system 201, and with external components such as external network elements, other machines, client devices, cloud computing services, etc. Communications may further include communicating with external devices via a network interface over a LAN, WAN, or the public Internet.

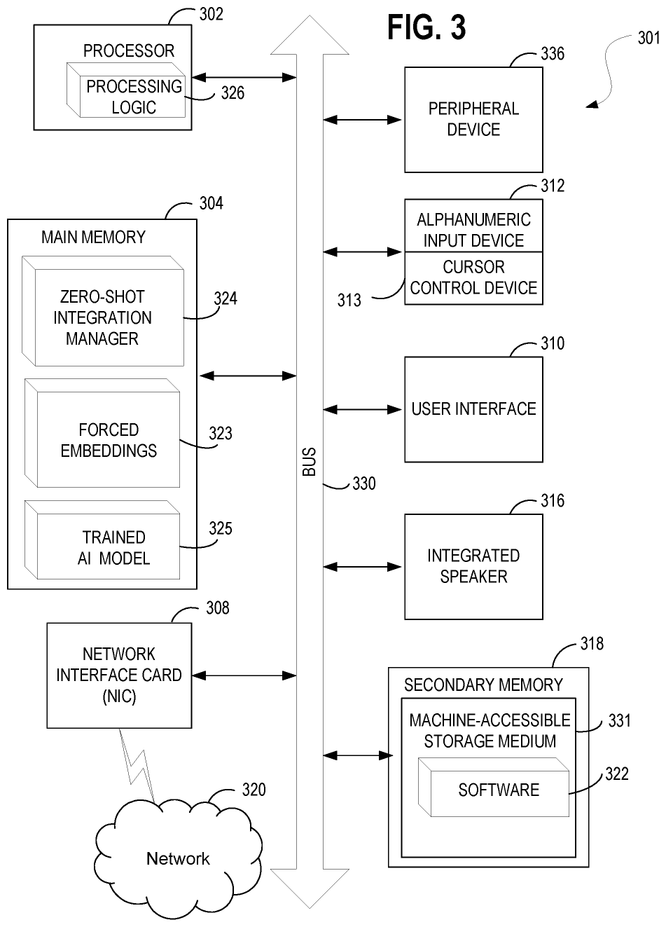

FIG. 3 illustrates a diagrammatic representation of a machine 301 in the exemplary form of a computer system, in accordance with one embodiment, within which a set of instructions, for causing the machine/computer system 301 to perform any one or more of the methodologies discussed herein, may be executed.

In alternative embodiments, the machine may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set-top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, while only a single machine is illustrated, the term “machine” shall also be taken to include any collection of machines (e.g., computers) that individually or jointly execute a set (or multiple sets) of instructions to perform any one or more of the methodologies discussed herein.

The exemplary computer system 301 includes a processor 302, a main memory 304 (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory 318 (e.g., a persistent storage device including hard disk drives and a persistent database and/or a multi-tenant database implementation), which communicate with each other via a bus 330. Main memory 304 includes a zero-shot integration manager 324 which provides input to a Convolutional Neural Network (CNN) communicably interfaced with the system, as well as forced embeddings 323 for using in training an AI model and ultimately a trained AI model 325 for utilization in evaluating medical image data in support of the methodologies and techniques described herein. Main memory 304 and its sub-elements are further operable in conjunction with processing logic 326 and processor 302 to perform the methodologies discussed herein.

Processor 302 represents one or more specialized and specifically configured processing devices such as a microprocessor, central processing unit, or the like. More particularly, the processor 302 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 302 may also be one or more special-purpose processing devices such as an application-specific integrated circuit (ASIC), a field programmable gate array (FPGA), a digital signal processor (DSP), network processor, or the like. Processor 302 is configured to execute the processing logic 326 for performing the operations and functionality which is discussed herein.

The computer system 301 may further include a network interface card 308. The computer system 301 also may include a user interface 310 (such as a video display unit, a liquid crystal display, etc.), an alphanumeric input device 312 (e.g., a keyboard), a cursor control device 313 (e.g., a mouse), and a signal generation device 316 (e.g., an integrated speaker). The computer system 301 may further include peripheral device 336 (e.g., wireless or wired communication devices, memory devices, storage devices, audio processing devices, video processing devices, etc.).

The secondary memory 318 may include a non-transitory machine-readable storage medium or a non-transitory computer readable storage medium or a non-transitory machine-accessible storage medium 331 on which is stored one or more sets of instructions (e.g., software 322) embodying any one or more of the methodologies or functions described herein. The software 322 may also reside, completely or at least partially, within the main memory 304 and/or within the processor 302 during execution thereof by the computer system 301, the main memory 304 and the processor 302 also constituting machine-readable storage media. The software 322 may further be transmitted or received over a network 320 via the network interface card 308.

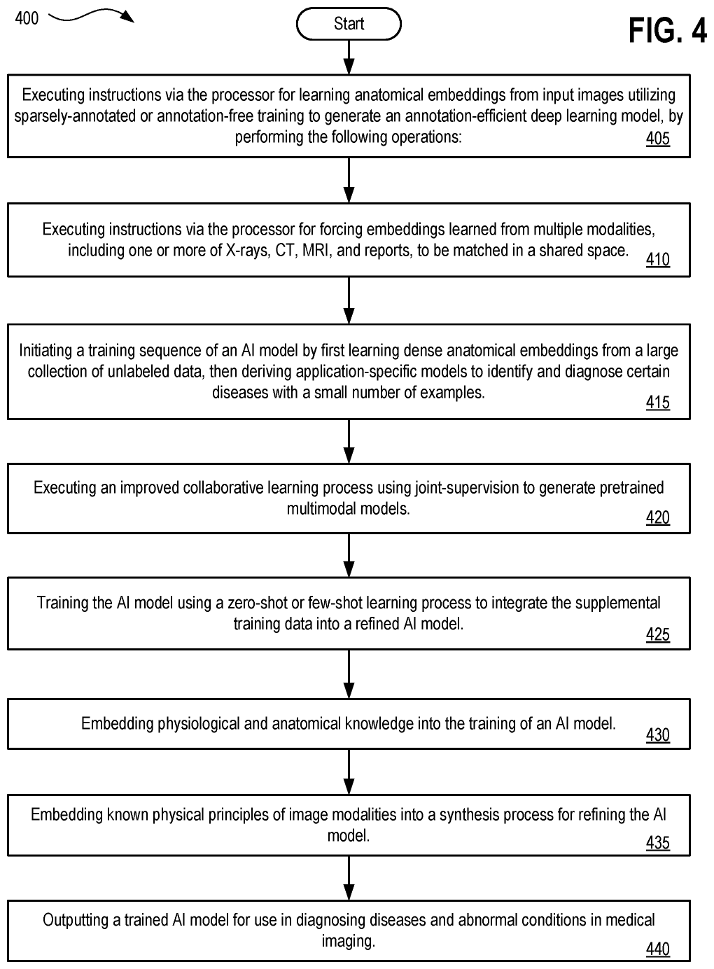

FIG. 4 depicts a flow diagram illustrating a method for implementing annotation-efficient deep learning models utilizing sparsely-annotated or annotation-free training, in which trained models are then utilized for the processing of medical imaging, in accordance with disclosed embodiments. Method 400 may be performed by processing logic that may include hardware (e.g., circuitry, dedicated logic, programmable logic, microcode, etc.), software (e.g., instructions run on a processing device) to perform various operations such as designing, defining, retrieving, parsing, persisting, exposing, loading, executing, operating, receiving, generating, storing, maintaining, creating, returning, presenting, interfacing, communicating, transmitting, querying, processing, providing, determining, triggering, displaying, updating, sending, etc., in pursuance of the systems and methods as described herein. Some of the blocks and/or operations listed below are optional in accordance with certain embodiments. The numbering of the blocks presented is for the sake of clarity and is not intended to prescribe an order of operations in which the various blocks must occur.

With reference to the method 400 depicted at FIG. 4, there is a method performed by a system specially configured to defend against adversarial attacks on neural networks. Such a system may be configured with at least a processor and a memory to execute specialized instructions which cause the system to learn anatomical embeddings from input images utilizing sparsely-annotated or annotation-free training to generate an annotation-efficient deep learning model (refer to block 405), by performing the following operations:

At block 410, processing logic executes instructions via the processor for forcing embeddings learned from multiple modalities, including one or more of X-rays, CT, MRI, and reports, to be matched in a shared space.

At block 415, processing logic initiates a training sequence of an AI model by first learning dense anatomical embeddings from a large collection of unlabeled data, then deriving application-specific models to identify and diagnose certain diseases with a small number of examples.

For instance, according to certain embodiments, stored instructions may specially configure the system to execute a method in which the system initiates a training sequence of an AI model by first learning of dense anatomical embeddings to train the AI model to mimic human abilities of learning to identify and diagnose certain diseases through the study of a small number of textbook examples.

At block 420, processing logic executes an improved collaborative learning process using joint-supervision to generate pretrained multimodal models.

For instance, according to certain embodiments, stored instructions may specially configure the system to execute a method in which the system executes an improved collaborative learning process using joint-supervision to generate supplemental training data in which joint supervision is used to create multiple supervision signals, which then in turn are used to pretrain the multimodal model.

At block 425, processing logic trains the AI model using a zero-shot or few-shot learning process to integrate the supplemental training data into a refined AI model.

For instance, according to certain embodiments, stored instructions may specially configure the system to execute a method in which the system trains the AI model using a zero-shot or few-shot learning process to integrate the supplemental training data generated from the improved collaborative learning process due to the application of the joint supervision which creates the multiple supervision signals for the sake of training to render the refined AI model.

At block 430, processing logic embeds physiological and anatomical knowledge into the training of an AI model.

At block 435, processing logic embeds known physical principles of image modalities into a synthesis process for refining the AI model.

At block 440, processing logic outputs a trained AI model for use in diagnosing diseases and abnormal conditions in medical imaging.

According to another embodiment of method 400, executing the improved collaborative learning process using joint-supervision comprises forcing embeddings learned from multiple modalities to be matched in a shared space.

According to another embodiment of method 400, the multiple modalities include one or more of: X-rays imaging; Computerized Tomography (CT) scans; Magnetic resonance imaging (MRI) scans; and text derived from medical charts and reports.

According to another embodiment of method 400, forcing the embeddings learned from the multiple modalities yields AI model refinements through improved collaborative learning via joint supervision and cross-supervision.

According to another embodiment, method 400 further includes: generating AI model refinements by executing a self-supervised learning operation, which includes at least: receiving as input diagnostic reports having embedded therein identification of objective disease conditions as determined by medical testing; automatically extracting the objective disease conditions from the diagnostic reports; and refining the AI model using the objective disease

According to another embodiment, method 400 further includes: generating AI model refinements by executing a self-supervised learning operation, which includes at least: receiving as input medical reports having embedded therein identification of subjective disease conditions as determined by medical experts having authored the medical reports; automatically extracting the subjective disease conditions from the medical reports; and refining the AI model using the subjective disease conditions extracted.

According to another embodiment, method 400 further includes: receiving as input subjective disease conditions extracted from medical reports; receiving as input objective disease conditions extracted from diagnostic reports; executing a self-supervised learning operation to refine the AI model using the subjective disease conditions and the objective disease conditions received as input; and outputting the trained AI model having the refinements from the self-supervised learning operation represented therein.

According to another embodiment, method 400 further includes: iteratively generating synthesized annotations by artificially rendering realistic-looking medical images based on ground truth information associated with known disease conditions of physiological and anatomical examples; aggregating the synthesized annotations into an artificial dataset for use in training AI models; and refining the AI model by training using the artificial dataset.

According to another embodiment of method 400, executing the improved collaborative learning process using joint-supervision comprises: receiving medical images augmented with clinical notes and diagnostic reports; learning generic semantic representations jointly from both the medical images received and the clinical notes and diagnostic reports associated with the medical images received through self-supervision learning; outputting the generic semantic representations learned as supplemental training refinements; and refining the AI model by training using the supplemental training refinements having the generic semantic representations embedded therein.

According to a particular embodiment, there is a non-transitory computer readable storage medium having instructions stored thereupon that, when executed by a system having at least a processor and a memory therein, the instructions cause the system to learn anatomical embeddings from input images utilizing sparse or empty training datasets to generate an annotation-efficient deep learning model, by performing operations including: executing instructions via the processor for forcing embeddings learned from multiple modalities, including one or more of X-rays, CT, MRI, and reports, to be matched in a shared space; initiating a training sequence of an AI model by first learning of dense anatomical embeddings to train the AI model to mimic human abilities of learning to identify and diagnose certain diseases through the study of a small number of textbook examples; executing an improved collaborative learning process using joint-supervision to generate supplemental training data; training the AI model using a zero-shot or few-shot learning process to integrate the supplemental training data generated from the improved collaborative learning process into a refined AI model; embedding physiological and anatomical knowledge into the training of an AI model; embedding known physical principles of image modalities into a synthesis process for refining the AI model; and outputting a trained AI model for use in diagnosing diseases and abnormal conditions in medical imaging.

While the subject matter disclosed herein has been described by way of example and in terms of the specific embodiments, it is to be understood that the claimed embodiments are not limited to the explicitly enumerated embodiments disclosed. To the contrary, the disclosure is intended to cover various modifications and similar arrangements as are apparent to those skilled in the art. Therefore, the scope of the appended claims is to be accorded the broadest interpretation so as to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

What is claimed is:

1. A system comprising:
a memory to store instructions;
a processor to execute the instructions stored in the memory;
wherein the system is specially configured to learn anatomical embeddings from input images utilizing sparsely-annotated or annotation-free training to generate an annotation-efficient deep learning model by performing the following operations:
executing instructions via the processor for forcing embeddings learned from multiple modalities, including one or more of X-rays, CT, MRI, and reports, to be matched in a shared space;
initiating a training sequence of an AI model by first learning dense anatomical embeddings from a large collection of unlabeled data, then deriving application-specific models to identify and diagnose certain diseases with a small number of examples;
executing an improved collaborative learning process using joint-supervision to generate pretrained multimodal models;
training the AI model using a zero-shot or few-shot learning process to integrate the supplemental training data into a refined AI model;
embedding physiological and anatomical knowledge into the training of an AI model;
embedding known physical principles of image modalities into a synthesis process for refining the AI model; and
outputting a trained AI model for use in diagnosing diseases and abnormal conditions in medical imaging.

2. The system of claim 1, wherein executing the improved collaborative learning process using joint-supervision comprises forcing embeddings learned from multiple modalities to be matched in a shared space.

3. The system of claim 2, wherein the multiple modalities include one or more of:
X-rays imaging;
Computerized Tomography (CT) scans;
Magnetic resonance imaging (MRI) scans; and
text derived from medical charts and reports.

4. The system of claim 2, wherein forcing the embeddings learned from the multiple modalities yields AI model refinements through improved collaborative learning via joint-supervision and cross-supervision.

5. The system of claim 1, further comprising:
generating AI model refinements by executing a self-supervised learning operation, which includes at least:
receiving as input diagnostic reports having embedded therein identification of objective disease conditions as determined by medical testing;
automatically extracting the objective disease conditions from the diagnostic reports; and
refining the AI model using the objective disease conditions extracted.

6. The system of claim 1, further comprising:
generating AI model refinements by executing a self-supervised learning operation, which includes at least:
receiving as input medical reports having embedded therein identification of subjective disease conditions as determined by medical experts having authored the medical reports;
automatically extracting the subjective disease conditions from the medical reports; and
refining the AI model using the subjective disease conditions extracted.

7. The system of claim 1, further comprising:
receiving as input subjective disease conditions extracted from medical reports;
receiving as input objective disease conditions extracted from diagnostic reports;
executing a self-supervised learning operation to refine the AI model using the subjective disease conditions and the objective disease conditions received as input; and
outputting the trained AI model having the refinements from the self-supervised learning operation represented therein.

8. The system of claim 1, further comprising:
iteratively generating synthesized annotations by artificially rendering realistic-looking medical images based on ground truth information associated with known disease conditions of physiological and anatomical examples;
aggregating the synthesized annotations into an artificial dataset for use in training AI models; and
refining the AI model by training using the artificial dataset.

9. The system of claim 1, wherein executing the improved collaborative learning process using joint-supervision comprises:
receiving medical images augmented with clinical notes and diagnostic reports;
learning generic semantic representations jointly from both the medical images received and the clinical notes and diagnostic reports associated with the medical images received through self-supervision learning;
outputting the generic semantic representations learned as supplemental training refinements; and
refining the AI model by training using the supplemental training refinements having the generic semantic representations embedded therein.

10. A method performed by a system having at least a processor and a memory therein to execute instructions for learning anatomical embeddings from input images utilizing sparsely-annotated or annotation-free training to generate an annotation-efficient deep learning model, wherein the method comprises:
executing instructions via the processor for forcing embeddings learned from multiple modalities, including one or more of X-rays, CT, MRI, and reports, to be matched in a shared space;
initiating a training sequence of an AI model by first learning dense anatomical embeddings from a large collection of unlabeled data, then deriving application-specific models to identify and diagnose certain diseases with a small number of examples;
executing an improved collaborative learning process using joint-supervision to generate pretrained multimodal models;
training the AI model using a zero-shot or few-shot learning process to integrate the supplemental training data into a refined AI model;
embedding physiological and anatomical knowledge into the training of an AI model;
embedding known physical principles of image modalities into a synthesis process for refining the AI model; and
outputting a trained AI model for use in diagnosing diseases and abnormal conditions in medical imaging.

11. The method of claim 10, wherein executing the improved collaborative learning process using joint-supervision comprises forcing embeddings learned from multiple modalities to be matched in a shared space.

12. The method of claim 11, wherein the multiple modalities include one or more of:
X-rays imaging;
Computerized Tomography (CT) scans;
Magnetic resonance imaging (MRI) scans; and
text derived from medical charts and reports.

13. The method of claim 11, wherein forcing the embeddings learned from the multiple modalities yields AI model refinements through improved collaborative learning via joint-supervision and cross-supervision.

14. The method of claim 10, further comprising:
generating AI model refinements by executing a self-supervised learning operation, which includes at least:
receiving as input diagnostic reports having embedded therein identification of objective disease conditions as determined by medical testing;
automatically extracting the objective disease conditions from the diagnostic reports; and
refining the AI model using the objective disease conditions extracted.

15. The method of claim 10, further comprising:
generating AI model refinements by executing a self-supervised learning operation, which includes at least:
receiving as input medical reports having embedded therein identification of subjective disease conditions as determined by medical experts having authored the medical reports;
automatically extracting the subjective disease conditions from the medical reports; and
refining the AI model using the subjective disease conditions extracted.

16. The method of claim 10, further comprising:
receiving as input subjective disease conditions extracted from medical reports;
receiving as input objective disease conditions extracted from diagnostic reports;
executing a self-supervised learning operation to refine the AI model using the subjective disease conditions and the objective disease conditions received as input; and
outputting the trained AI model having the refinements from the self-supervised learning operation represented therein.

17. The method of claim 10, further comprising:
iteratively generating synthesized annotations by artificially rendering realistic-looking medical images based on ground truth information associated with known disease conditions of physiological and anatomical examples;
aggregating the synthesized annotations into an artificial dataset for use in training AI models; and
refining the AI model by training using the artificial dataset.

18. The method of claim 10, wherein executing the improved collaborative learning process using joint-supervision comprises:
receiving medical images augmented with clinical notes and diagnostic reports;
learning generic semantic representations jointly from both the medical images received and the clinical notes and diagnostic reports associated with the medical images received through self-supervision learning;
outputting the generic semantic representations learned as supplemental training refinements; and
refining the AI model by training using the supplemental training refinements having the generic semantic representations embedded therein.

19. Non-transitory computer-readable storage media having instructions stored thereupon that, when executed by a system having at least a processor and a memory therein, the instructions cause the system to learn anatomical embeddings from input images utilizing sparsely-annotated or annotation-free training to generate an annotation-efficient deep learning model, by performing operations including:
executing instructions via the processor for forcing embeddings learned from multiple modalities, including one or more of X-rays, CT, MRI, and reports, to be matched in a shared space;
initiating a training sequence of an AI model by first learning dense anatomical embeddings from a large collection of unlabeled data, then deriving application-specific models to identify and diagnose certain diseases with a small number of examples;
executing an improved collaborative learning process using joint-supervision to generate pretrained multimodal models;
training the AI model using a zero-shot or few-shot learning process to integrate the supplemental training data into a refined AI model;
embedding physiological and anatomical knowledge into the training of an AI model;
embedding known physical principles of image modalities into a synthesis process for refining the AI model; and
outputting a trained AI model for use in diagnosing diseases and abnormal conditions in medical imaging.

20. The non-transitory computer-readable storage media of claim 19, wherein executing the improved collaborative learning process using joint-supervision comprises:
receiving medical images augmented with clinical notes and diagnostic reports;
learning generic semantic representations jointly from both the medical images received and the clinical notes and diagnostic reports associated with the medical images received through self-supervision learning;
outputting the generic semantic representations learned as supplemental training refinements; and
refining the AI model by training using the supplemental training refinements having the generic semantic representations embedded therein.
